(19) United States
(12) Patent Application Publication
(10) Pub. No.: US 2007/0135690 A1
(43) Pub. Date: Jun. 14, 2007

(54) Mobile communication device that provides health feedback

(76) Inventor: Richard Vincent Nicholl, Raleigh, NC (US)

Correspondence Address: HARRITY SNYDER, L.L.P., Suite 600, 11350 Random Hills Road, Fairfax, VA, 22030, US

(21) Appl. No.: 11/296,370

(22) Filed: Dec. 8, 2005

(51) Int. Cl. A61B 5/00; A63B 71/00; A61B 5/02

(52) U.S. Cl. 600/301; 600/485; 600/500; 128/905; 482/8

(57) ABSTRACT

A mobile communication device obtains health-related information associated with a user of the mobile communication device, determines whether a health goal has been reached based on the health-related information, provides negative feedback to the user when the health goal has not been reached, and provides positive feedback to the user when the health goal has been reached.

### BACKGROUND

1. Field of the Invention

Implementations described herein relate generally to mobile communication devices and, more particularly, to a mobile communication device that provides health feedback.

2. Description of Related Art

Individuals are constantly trying to improve their health. They regularly begin diet and exercise programs. Oftentimes, the diet and exercise programs fail within a few weeks or a few months. The main reason that these programs fail is that the individuals lack the feedback that they need to continue on the programs.

### SUMMARY

According to one aspect, a method performed by a mobile communication device may include obtaining health-related information associated with a user of the mobile communication device; determining whether a health goal has been reached based on the health-related information; providing negative feedback to the user when the health goal has not been reached; and providing positive feedback to the user when the health goal has been reached.

According to another aspect, a mobile communication device may include means for obtaining real time health-related information associated with a user of the mobile communication device; means for setting a threshold; means for determining whether the health-related information exceeds the threshold; means for providing negative feedback to the user when the health-related information does not exceed the threshold; and means for providing positive feedback to the user when the health-related information exceeds the threshold.

### BRIEF DESCRIPTION OF THE DRAWINGS

The accompanying drawings, which are incorporated in and constitute a part of this specification, illustrate an embodiment of the invention and, together with the description, explain the invention. In the drawings,

FIG. 1 is a diagram of an exemplary mobile communication device according to an implementation consistent with the principles of the invention;

FIG. 2 is a diagram of exemplary components of the mobile communication device of FIG. 1;

FIG. 3 is a flowchart of an exemplary process for providing health feedback;

FIGS. 4 and 5 are exemplary diagrams of visual feedback that may be provided;

FIG. 6 is an exemplary diagram of varying degrees of feedback that may be provided;

FIG. 7 is a flowchart of an exemplary implementation consistent with the principles of the invention; and

FIGS. 8 and 9 are exemplary diagrams of feedback that may be provided for the exemplary implementation of FIG. 7.

### DETAILED DESCRIPTION

The following detailed description of the invention refers to the accompanying drawings. The same reference numbers in different drawings may identify the same or similar elements. Also, the following detailed description does not limit the invention.

The description to follow will describe a mobile communication device. As used herein, a “mobile communication device” may include a radiotelephone; a personal communications system (PCS) terminal that may combine a cellular radiotelephone with data processing, a facsimile, and data communications capabilities; a personal digital assistant (PDA) that can include a radiotelephone, pager, Internet/intranet access, web browser, organizer, calendar, and/or global positioning system (GPS) receiver; and a laptop and/or palmtop receiver or other appliance that includes a radiotelephone transceiver.

### Exemplary Mobile Communication Device

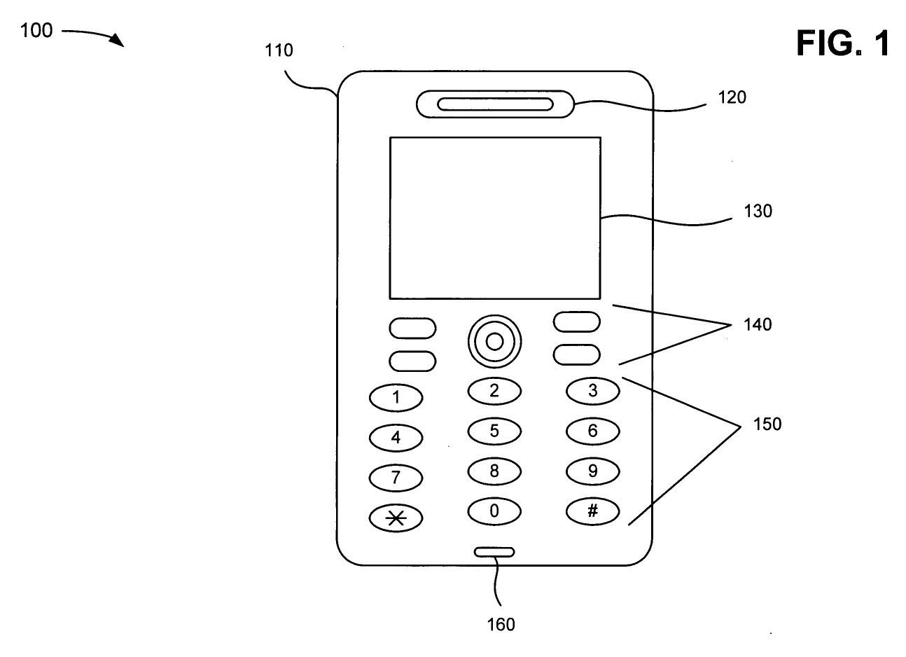

FIG. 1 is a diagram of an exemplary mobile communication device 100 according to an implementation consistent with the principles of the invention. As shown in FIG. 1, device 100 may include a housing 110, a speaker 120, a display 130, control buttons 140, a keypad 150, and a microphone 160. Housing 110 may protect the components of device 100 from outside elements. Speaker 120 may provide audible information to a user of device 100. Display 130 may provide visual information to the user. For example, display 130 may provide information regarding incoming or outgoing calls, games, phone books, the current time, etc. In an implementation consistent with the principles of the invention, display 130 may provide the user with health feedback information. Control buttons 140 may permit the user to interact with device 100 to cause device 100 to perform one or more operations. Keypad 150 may include a standard telephone keypad. Microphone 160 may receive audible information from the user.

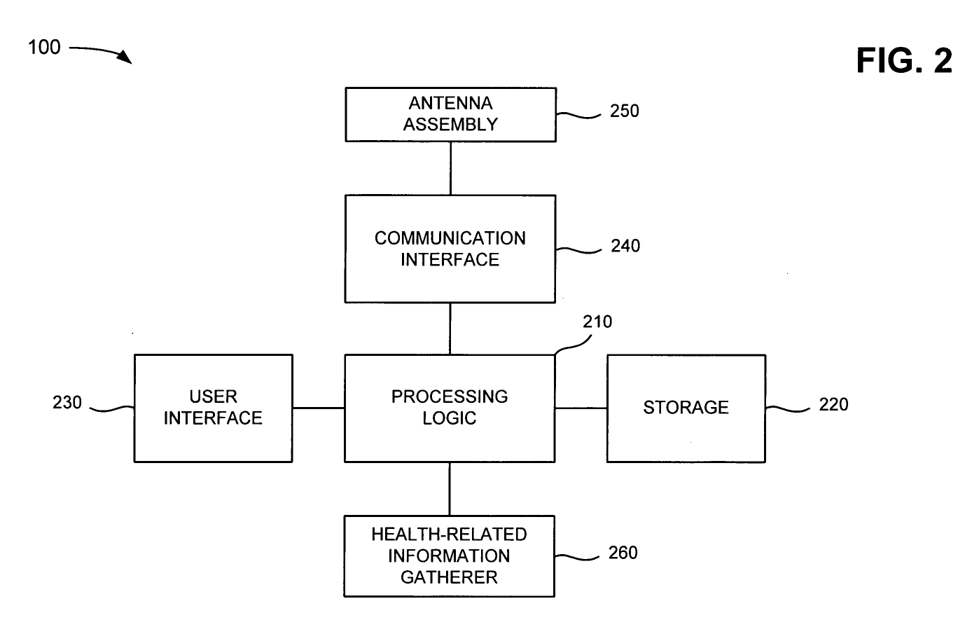

FIG. 2 is a diagram of exemplary components of mobile communication device 100. As shown in FIG. 2, device 100 may include processing logic 210, storage 220, user interface 230, communication interface 240, antenna assembly 250, and health-related information gatherer 260. Processing logic 210 may include a processor, microprocessor, an application specific integrated circuit (ASIC), field programmable gate array (FPGA), or the like. Processing logic 210 may include data structures or software programs to control operation of device 100 and its components. Storage 220 may include a random access memory (RAM), a read only memory (ROM), and/or another type of memory to store data and instructions that may be used by processing logic 210.

User interface 230 may include mechanisms for inputting information to device 100 and/or for outputting information from device 100. Examples of input and output mechanisms might include a speaker (e.g., speaker 120) to receive electrical signals and output audio signals, a microphone (e.g., microphone 160) to receive audio signals and output electrical signals, buttons (e.g., control buttons 140 and/or keys of keypad 150) to permit data and control commands to be input into device 100, a display (e.g., display 130) to output visual information, and/or a vibrator to cause device 100 to vibrate.

Communication interface 240 may include, for example, a transmitter that may convert baseband signals from processing logic 210 to radio frequency (RF) signals and/or a receiver that may convert RF signals to baseband signals. Alternatively, communication interface 240 may include a transceiver to perform functions of both a transmitter and a receiver. Communication interface 240 may connect to antenna assembly 250 for transmission and reception of the RF signals. Antenna assembly 250 may include one or more antennas to transmit and receive RF signals over the air. Antenna assembly 250 may receive RF signals from communication interface 240 and transmit them over the air and receive RF signals over the air and provide them to communication interface 240.

Health-related information gatherer 260 may obtain health-related information from a user. In one implementation, the health-related information may correspond to the amount of exercise that the user gets during a time period. In this case, health-related information gatherer 260 may include a pedometer that may use pendulum technology, an accelerometer, and/or electronics to count the number of footsteps a user takes over a period of time. Alternatively, health-related information gatherer 260 may include global positioning system (GPS) technology to track the distance traveled by the user. In another implementation, the health-related information may correspond to the user's heart rate, blood pressure, and/or body fat. In this case, health-related information gatherer 260 may include a sensor to obtain a reading of the user's heart rate, blood pressure, and/or body fat. In yet another implementation, the health-related information may correspond to dietary information associated with the user. In this case, health-related information gatherer 260 may include a keyboard or another type of input buttons (e.g., control buttons 140 and/or keys of keypad 150) that permits the user to enter information regarding what the user has eaten.

As will be described in detail below, mobile communication device 100, consistent with the principles of the invention, may perform certain operations relating to the providing of health feedback. Device 100 may perform these operations in response to processing logic 210 executing software instructions of a health feedback application contained in a computer-readable medium, such as storage 220. A computer-readable medium may be defined as a physical or logical memory device and/or carrier wave.

The software instructions may be read into storage 220 from another computer-readable medium or from another device via communication interface 240. The software instructions contained in storage 220 may cause processing logic 210 to perform processes that will be described later. Alternatively, hardwired circuitry may be used in place of or in combination with software instructions to implement processes consistent with the principles of the invention. Thus, implementations consistent with the principles of the invention are not limited to any specific combination of hardware circuitry and software.

### Exemplary Processing

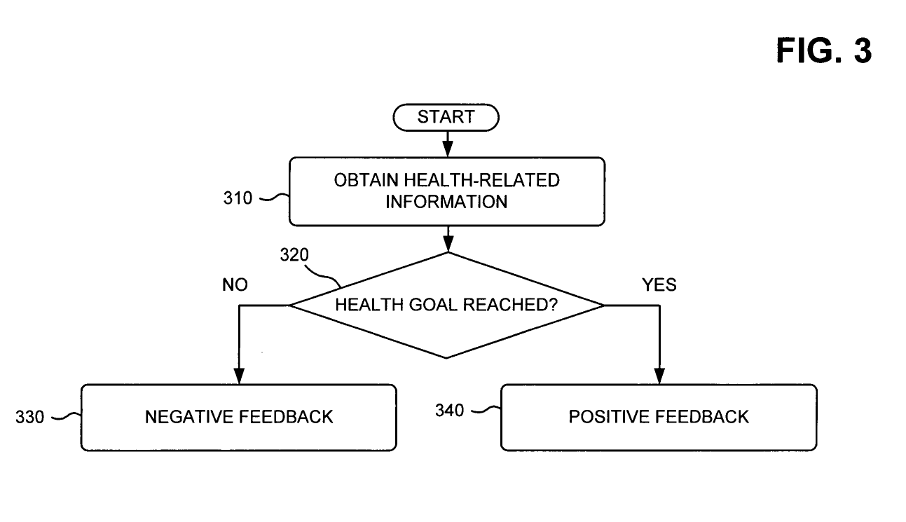

FIG. 3 is a flowchart of an exemplary process for providing health feedback. Processing may begin with the starting of a health feedback application on device 100. In one implementation, the health feedback application may be started when instructed by a user of device 100. In another implementation, the health feedback application may be started automatically, such as when device 100 powers on.

Health-related information may be obtained (block 310). As explained above, the health-related information may corresponding to a count of the number of footsteps taken by the user, a determination of the distance traveled by the user, a reading of the user's heart rate, blood pressure, and/or body fat, and/or dietary information. The health-related information may be regularly or periodically obtained. For example, health-related information gatherer 260 may count the user's footsteps as the user walks throughout the day. Alternatively, health-related information gatherer 260 may read the user's heart rate when instructed by the user.

It may be determined whether a health goal has been reached (block 320). With regard to footsteps taken or distance traveled, processing logic 210 may determine whether the number of footsteps taken by the user and/or the distance traveled by the user over a predetermined time period matches or exceeds a threshold. With regard to heart rate, blood pressure, and/or body fat, processing logic 210 may determine whether the user's heart rate, blood pressure, and/or body fat readings are within one or more thresholds. With regard to dietary information, processing logic 210 may determine whether the user's food intake has not exceeded one or more thresholds and/or satisfies one or more other thresholds.

The threshold may be set by the user or automatically set based on general health guidelines. For example, general health guidelines might indicate that a user should walk 10,000 steps a day or travel at least approximately 8 kilometers a day. General health guidelines might indicate healthy readings for the resting heart rate (e.g., 60-100 beats per minute), blood pressure (e.g., below 120/80), and body fat (e.g., 20-22%). General health guidelines might indicate that a user should not exceed 1600 to 2800 calories a day and/or a user should get certain amounts of protein, vitamins, thiamin, riboflavin, niacin, folacin, calcium, phosphorus, magnesium, iron, zinc, iodine, and/or selenium per day.

The health goal may be periodically reset. For example, the health goal might be set on a daily, weekly, or monthly basis. In this case, processing logic 210 may use an internal clock and/or calendar of device 100 to determine when to reset the health goal. With regard to footsteps taken or distance traveled, processing logic 210 may reset the health goal on a daily or weekly basis. With regard to heart rate, blood pressure, and/or body fat, processing logic 210 may reset the health goal on a monthly or semi-monthly basis. With regard to dietary information, processing logic 210 may reset the health goal on a daily basis.

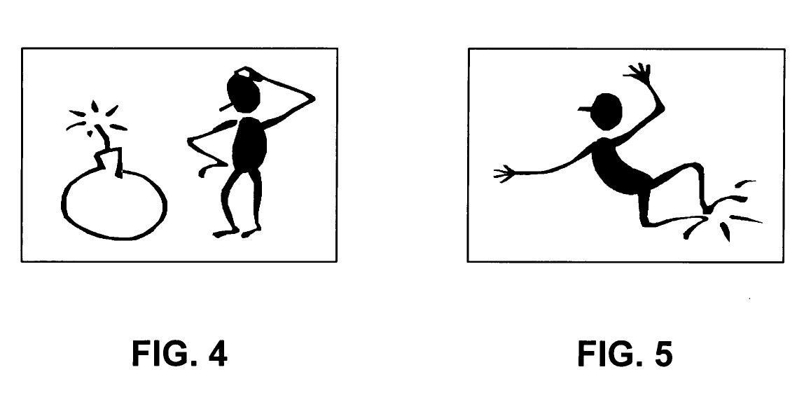

When the health goal has not been reached, the user may be given negative feedback (block 330). The negative feedback may be audible, visual, and/or physical. Negative audible feedback might include the playing of a sound that connotes a negative tone or message, such as a voice, a dog barking or growling, or a certain musical composition. Negative visual feedback might include an image or video that connotes a negative tone or message, such as an image or video of a sad or angry person or an image or video of a dog barking meanly or growling. FIG. 4 is a diagram of negative visual feedback that may be presented to the user. In this case, the negative visual feedback corresponds to a sad person standing next to a bomb that is about to explode. Negative physical feedback might include a vibration of device 100.

When the health goal has been reached, the user may be given positive feedback (block 340). Like the negative feedback, the positive feedback may be audible, visual, and/or physical. Positive audible feedback might include the playing of a sound that connotes a positive tone or message, such as a voice, a dog happily barking, or a certain musical composition. Positive visual feedback might include an image or video that connotes a positive tone or message, such as an image or video of a happy person or an image or video of a dog wagging its tail. FIG. 5 is a diagram of positive visual feedback that may be presented to the user. In this case, the positive visual feedback corresponds to a happy person who is jumping in the air and clicking his heels together. Positive physical feedback might include a vibration of device 100, which may differ from any negative physical feedback that may be given to the user.

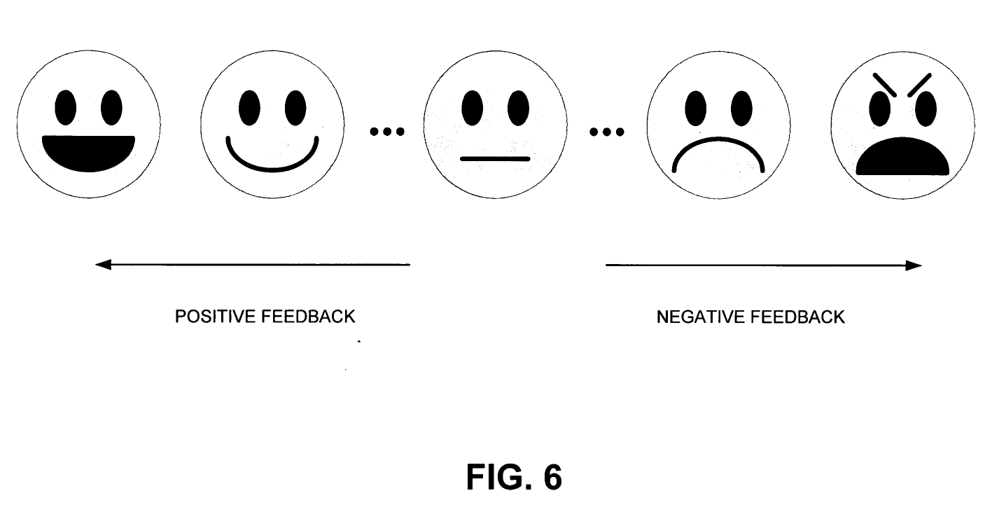

Instead of providing negative feedback when the user does not reach the health goal and positive feedback when the user reaches the health goal, processing logic 210 may provide various degrees of feedback. FIG. 6 is an exemplary diagram of varying degrees of visual feedback that may be provided. As shown in FIG. 6, the face may get happier as the user approaches and finally reaches the health goal and angrier as the user falls further away from the health goal. For example, if the user has a daily goal, the face may get angrier as the day progresses and the user is not close to meeting the health goal.

### EXAMPLE

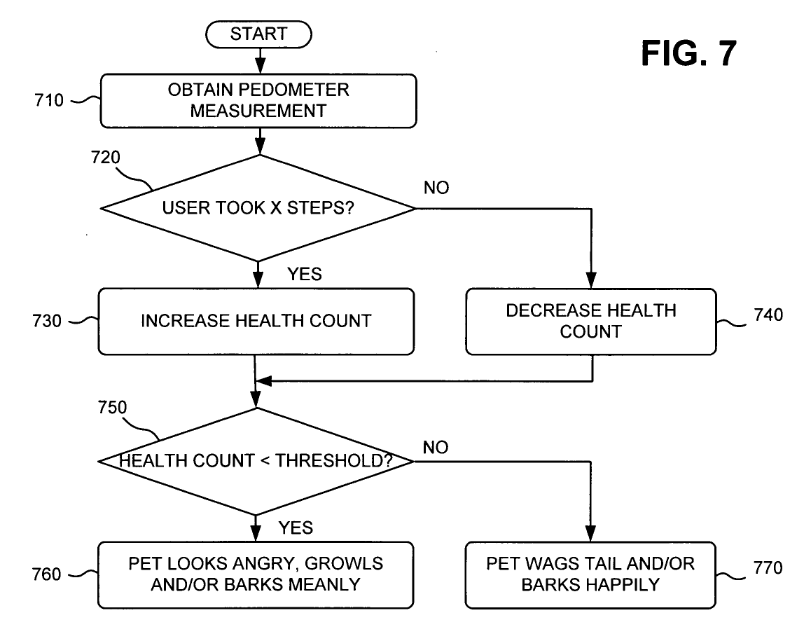

FIG. 7 is a flowchart of an exemplary implementation consistent with the principles of the invention. Assume, for this implementation, that health-related information gatherer 260 takes the form of a pedometer that counts the number of footsteps that a user takes. The processing of FIG. 7 may begin with the starting of the health feedback application on device 100. As explained above, the health feedback application may be started automatically when device 100 powers on or may be started when instructed by a user of device 100. In this case, assume that the health feedback application takes the form of a healthy pet that resides on device 100 and provides feedback to the user on the progress of the user toward the health goal.

Once the health feedback application is started, pedometer measurements may be obtained as the user walks (block 710). It may be periodically determined whether the user has taken a predetermined number (“X”) of steps (block 720). The predetermined number of steps may be set equal to or less than the health goal. The determination of block 720 may be based on certain time periods. For example, it may be determined whether the user has taken a predetermined number of steps in the period from 9:00 a.m. to 10:00 a.m. or from 9:00 a.m. to 12:00 p.m.

If the user has taken more than the predetermined number of steps, then a health count value (i.e., some measure of the user's health or progress toward the health goal, which may initially be set to zero and/or periodically reset to zero) may be increased (block 730). If the user has not taken more than the predetermined number of steps, then the health count value may be decreased (block 740).

It may be determined whether the health count value is greater than a threshold (block 750). In a simple case where the predetermined number equals the health goal, then the threshold may be set equal to zero so that an increase in the health count value increases the health count value over the threshold and a decrease in the health count value decreases the health count value below the threshold.

In a more complex case, the determination of whether the health count value is greater than the threshold may be performed periodically such that blocks 710-740 may occur multiple times for each occurrence of block 750. In this case, the predetermined number of steps may be set to a value less than the health goal and the threshold may be set to equal the health goal.

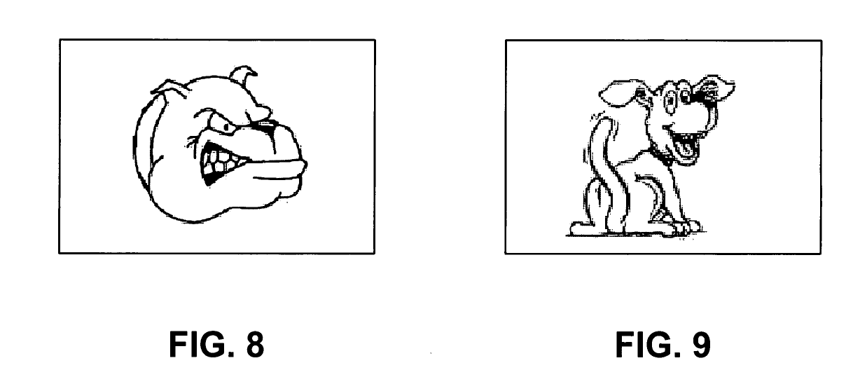

When the health count value is less than the threshold, then the user may be provided with negative feedback, such as the pet looking angry, growling, and/or barking meanly (block 760). FIG. 8 is a diagram of negative feedback that may be presented to the user. In this case, the negative feedback corresponds to an angry pet that may growl and/or bark meanly.

When the health count value is greater than the threshold, then the user may be provided with positive feedback, such as the pet wagging its tail and/or barking happily (block 770). FIG. 9 is a diagram of positive feedback that may be presented to the user. In this case, the positive feedback corresponds to a happy pet that may wag its tail and/or bark happily.

### CONCLUSION

Implementations consistent with the principles of the invention may correspond to a mobile communication device that provides health feedback to its user based on real-time data collected in association with the user.

The foregoing description of preferred embodiments of the invention provides illustration and description, but is not intended to be exhaustive or to limit the invention to the precise form disclosed. Modifications and variations are possible in light of the above teachings or may be acquired from practice of the invention.

For example, while series of acts have been described with regard to FIGS. 3 and 7, the order of the acts may be modified in other implementations consistent with the principles of the invention. Further, non-dependent acts may be performed in parallel.

It will be apparent to one of ordinary skill in the art that aspects of the invention, as described above, may be implemented in many different forms of software, firmware, and hardware in the implementations illustrated in the figures. The actual software code or specialized control hardware used to implement aspects consistent with the principles of the invention is not limiting of the invention. Thus, the operation and behavior of the aspects were described without reference to the specific software code—it being understood that one of ordinary skill in the art would be able to design software and control hardware to implement the aspects based on the description herein.

No element, act, or instruction used in the present application should be construed as critical or essential to the invention unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

What is claimed is:

1. A method performed by a mobile communication device, comprising:
obtaining health-related information associated with a user of the mobile communication device;
determining whether a health goal has been reached based on the health-related information;
providing negative feedback to the user when the health goal has not been reached; and
providing positive feedback to the user when the health goal has been reached.

2. The method of claim 1, wherein the health-related information corresponds to a number of footsteps taken by the user; and
wherein obtaining health-related information associated with a user of the mobile communication device includes:
counting the number of footsteps taken by the user during a predetermined time period.

3. The method of claim 2, wherein determining whether a health goal has been reached includes:
determining whether the counted number of footsteps during the predetermined time period exceeds a threshold.

4. The method of claim 1, wherein the health-related information corresponds to a distance traveled by the user; and
wherein obtaining health-related information associated with a user of the mobile communication device includes:
determining the distance traveled by the user during a predetermined time period using global positioning system (GPS) information.

5. The method of claim 4, wherein determining whether a health goal has been reached includes:
determining whether the distance traveled by the user during the predetermined time period exceeds a threshold.

6. The method of claim 1, wherein the health-related information corresponds to at least one of a heart rate, blood pressure, or body fat associated with the user; and
wherein obtaining health-related information associated with a user of the mobile communication device includes:
taking a sensor reading of the at least one of a heart rate, blood pressure, or body fat associated with the user.

7. The method of claim 6, wherein determining whether a health goal has been reached includes:
determining whether the at least one of a heart rate, blood pressure, or body fat associated with the user exceeds one or more thresholds.

8. The method of claim 1, wherein the health-related information corresponds to dietary information associated with the user; and
wherein obtaining health-related information associated with a user of the mobile communication device includes:
obtaining information regarding food intake associated with the user during a predetermined time period.

9. The method of claim 8, wherein determining whether a health goal has been reached includes:
determining whether the food intake associated with the user does not exceed one or more first thresholds or satisfies one or more second thresholds during the predetermined time period.

10. The method of claim 1, wherein the health-related information corresponds to at least one of a number of footsteps taken by the user, a distance traveled by the user, a heart rate associated with the user, a blood pressure associated with the user, a body fat associated with the user, or dietary information associated with the user; and
wherein obtaining health-related information associated with a user of the mobile communication device includes:
counting the number of footsteps taken by the user when the health-related information corresponds to the number of footsteps taken by the user,
determining the distance traveled by the user using global positioning system (GPS) information when the health-related information corresponds to the distance traveled by the user,
taking a sensor reading of the heart rate associated with the user when the health-related information corresponds to the heart rate associated with the user,
taking a sensor reading of the blood pressure associated with the user when the health-related information corresponds to the blood pressure associated with the user,
taking a sensor reading of the body fat associated with the user when the health-related information corresponds to the body fat associated with the user, and
obtaining information regarding food intake associated with the user when the health-related information corresponds to dietary information associated with the user.

11. The method of claim 1, wherein obtaining health-related information associated with a user of the mobile communication device includes:
regularly obtaining the health-related information.

12. The method of claim 1, wherein determining whether a health goal has been reached includes:
setting a threshold, and
determining whether the health-related information exceeds the threshold.

13. The method of claim 12, wherein setting the threshold includes:
receiving information concerning the threshold from the user.

14. The method of claim 12, wherein setting the threshold includes:
automatically identifying the threshold based on general health guidelines.

15. The method of claim 12, wherein determining whether a health goal has been reached further includes:
periodically resetting the threshold.

16. The method of claim 1, wherein providing negative feedback to the user includes:
providing at least one of negative audible feedback, negative visual feedback, or negative physical feedback to the user.

17. The method of claim 1, wherein providing positive feedback to the user includes:
providing at least one of positive audible feedback, positive visual feedback, or positive physical feedback to the user.

18. The method of claim 1, wherein providing negative feedback to the user and providing positive feedback to the user in combination includes:
providing varying degrees of positive and negative feedback to the user based on how close the user is to reaching the health goal during a predetermined time period.

19. A mobile communication device, comprising:
means for obtaining real time health-related information associated with a user of the mobile communication device;
means for setting a threshold;
means for determining whether the health-related information meets or exceeds the threshold;
means for providing negative feedback to the user when the health-related information does not meet or exceed the threshold; and
means for providing positive feedback to the user when the health-related information meets or exceeds the threshold.

20. A mobile communication device, comprising:
a health-related information gatherer to obtain health-related information associated with a user of the mobile communication device; and
processing logic to:
determine whether a health goal has been reached based on the health-related information,
provide negative feedback to the user when the health goal has not been reached, and
provide positive feedback to the user when the health goal has been reached.

21. The mobile communication device of claim 20, wherein the health-related information gatherer includes at least one of:
a pedometer to count a number of footsteps taken by the user,
a global positioning system (GPS) unit to determine a distance traveled by the user,
a heart rate sensor to take a reading of a heart rate associated with the user,
a blood pressure sensor to take a reading of blood pressure associated with the user,
a body fat sensor to take a reading of body fat associated with the user, or
a set of buttons to obtain information regarding food intake associated with the user.

22. The mobile communication device of claim 21, wherein when determining whether a health goal has been reached, the processing logic is configured to determine whether the counted number of footsteps during a predetermined time period exceeds a threshold when the health-related information gatherer includes the pedometer.

23. The mobile communication device of claim 21, wherein when determining whether a health goal has been reached, the processing logic is configured to determine whether the distance traveled by the user during a predetermined time period exceeds a threshold when the health-related information gatherer includes the GPS unit.

24. The mobile communication device of claim 21, wherein when determining whether a health goal has been reached, the processing logic is configured to determine whether the heart rate associated with the user exceeds a threshold when the health-related information gatherer includes the heart rate sensor.

25. The mobile communication device of claim 21, wherein when determining whether a health goal has been reached, the processing logic is configured to determine whether the blood pressure associated with the user exceeds a threshold when the health-related information gatherer includes the blood pressure sensor.

26. The mobile communication device of claim 21, wherein when determining whether a health goal has been reached, the processing logic is configured to determine whether the body fat associated with the user exceeds a threshold when the health-related information gatherer includes the body fat sensor.

27. The mobile communication device of claim 21, wherein when determining whether a health goal has been reached, the processing logic is configured to determine whether the food intake associated with the user does not exceed one or more first thresholds or satisfies one or more second thresholds during a predetermined time period when the health-related information gatherer includes the set of buttons.

28. The mobile communication device of claim 20, wherein when determining whether a health goal has been reached, the processing logic is configured to:
set a threshold, and
determine whether the health-related information exceeds the threshold.

29. The mobile communication device of claim 28, wherein when setting the threshold, the processing logic is configured to receive information concerning the threshold from the user.

30. The mobile communication device of claim 28, wherein when setting the threshold, the processing logic is configured to automatically identify the threshold based on general health guidelines.

31. The mobile communication device of claim 28, wherein when determining whether a health goal has been reached, the processing logic is further configured to periodically reset the threshold.

32. The mobile communication device of claim 20, wherein when providing negative feedback to the user, the processing logic is configured to provide at least one of negative audible feedback, negative visual feedback, or negative physical feedback to the user.

33. The mobile communication device of claim 20, wherein when providing positive feedback to the user, the processing logic is configured to provide at least one of positive audible feedback, positive visual feedback, or positive physical feedback to the user.

34. The mobile communication device of claim 20, wherein when providing negative feedback to the user and providing positive feedback to the user, the processing logic is configured to provide varying degrees of positive and negative feedback to the user based on how close the user is to reaching the health goal during a predetermined time period.
